(19) United States
(12) Patent Application Publication
(10) Pub. No.: US 2003/0147889 A1
(43) Pub. Date: Aug. 7, 2003

(54) Antagonist of th-1 immunerresponse inducing cytokine for the treatment of autoimmune diseases

(76) Inventor: Michael Gerard Tovey, Paris (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/182,062

(22) PCT Filed: Jan. 25, 2001
(86) PCT No.: PCT/GB01/00285

(30) Foreign Application Priority Data
Jan. 25, 2000 (GB) 0001710.3

(51) Int. Cl. A61K 39/395

(52) U.S. Cl. 424/145.1

(57) ABSTRACT

Oromucosal administration of antagonists of cytokines associated with stimulation or enhancement of T helper 1 cell responses, preferably for example a Type 1-interferon antibody, is disclosed for inhibition of prevention of autoimmune diseases.

The present invention relates to therapeutic treatment based on use of antagonists of cytokines associated with stimulation or enhancement of T helper 1 (Th 1) cell responses. In particular, it relates to administration of such antagonists, referred to below as Th 1 cytokine antagonists, via the oromucosal route. In one embodiment, the invention relates to oromucosal administration of a Type 1-interferon antibody for inhibition or treatment of an autoimmune disease.

### BACKGROUND TO THE INVENTION

Th 1 cells and T helper 2 (Th 2) cells are functionally distinct subsets of active CD4⁺ T cells characterised by the pattern of cytokines that they produce. Thus, mouse Th 1 cells have been shown to produce interferon-γ (IFN-γ), tumour necrosis factor-β (TNF-β) and interleukin-2 (IL-2) whereas mouse Th 2 cells produce IL-4, IL-5, IL-6, IL-9, IL-10 and IL-13. Human Th 1 and Th 2 cells have similar patterns of cytokine secretion, although the synthesis of IL-2, IL-6, IL-10 and IL-13 is not as tightly restricted to a single subset of Th cells as in mouse. Th 1 and Th 2 cells not only produce different sets of cytokines but also preferentially express different activation markers. Thus, human Th 1 cells preferentially express lymphocyte activation gene 3 (LAG-3) while human Th 2 cells preferentially express CD 30, a member of the TNF receptor family. Th 1 cells also express E-selectin, p-selectin and the β-chain of the IL-12 receptor, which are not expressed by Th 2 cells.

The results of numerous studies suggest that Th 1 overreactivity plays a key role in the pathogenesis of autoimmune disease. As Th 1 cytokines and Th2 cytokines are mutually inhibitory for the differentiation and effector functions of Th 2 cells and Th1 cells respectively, the use of Th 2 cytokines or Th 1 cytokine antagonists can be expected to be an effective means of treating autoimmune disease. Indeed, systemic administration of the Th 2 cytokine IL-10 has previously been shown to be effective in reversing a Th1 cell response by increasing IL-4 and IL-5 production and reducing IL-2 and IFN-γ production. IL-2 and IFN-γ have previously been recognised as important cytokines controlling Th 1 cell responsiveness. IL-12 has also been shown to induce a Th 1 cell response (Sergio Romagnani, 1997, Immunology Today 18, 263-266; “The Th1/Th2 paradigm”).

IFN-α is also known to be produced during the course of Th 1 cell activation and a Th 1 response has been shown to play a determining role in the antiviral activity of IFN-α2 in patients infected with human papillomavirus (Arany and Tyring, J. Interferon and Cytokine Res. 1996, 16, 453). Abnormal production of IFN-α has additionally been reported to be associated with a number of autoimmune diseases including systemic lupus erythematosus (Shiozawa et al. Arthr. & Rheum. 1992, 35, 417), rheumatoid arthritis (Hopkins & Meager, Clin. Exp. Immunol. 1988, 73, 88) type 1 diabetes (Stewart et al. Science 1993, 260, 1942; Huang et al. Cell 1994, 1, 469), multiple sclerosis (Degre et al. Acta Neurol. Scand. 1976, 53,152) and psoriasis (Schmid et al. J. Interferon Res. 1994, 14, 229). A number of clinical reports have also described the development of the symptoms of autoimmune disease or the exacerbation of underlying autoimmune disease in patients treated with recombinant IFN-α2 (see, for example, Wada et al. Am. J. Gastroenterol. 1995, 90, 1366 and Perez et al. Am. J. Hematol. 1995, 49, 365).

IFN-α has also been implicated in the etiology of a number of other diseases. Thus, in AIDS patients a direct correlation has been reported between the level of circulating IFN-α and disease progression (Mildvan et al. The Lancet 1992, 339, 353). The results of other studies suggest that IFN-α also plays an important role in allograft rejection (Afifi et al. J. Immunol. 1985, 134, 3739) and in graft-versus-host-disease (Cleveland et al. Cell. Immunol. 1987, 110, 120).

IFN-α and IFN-β are known as Type 1-interferon (Type 1-IFN) and exert their characteristic biological activities by interaction with the Type 1-IFN receptor which is a heterodimer composed of two polypeptide chains, IFNAR1 and IFNAR2. The corresponding cDNAs have been cloned (Uze et al. Cell 1990, 60, 224-234 and Novick et al. Cell 1994, 77, 391-400).

Following binding of a Type 1-IFN to its cell surface receptor three STAT (Signal Transducers and Activators of Transcription) proteins, STAT1 (p91), STAT 1 beta (p84) and STAT2 (p113) are phosphorylated by the tyrosine kinases Jak-1 and Tyk-2 and form a complex denominated ISGF3. This is considered to constitute one of the first steps in the transmission of the interferon signal to the nucleus. The Tyk-2 kinase and the STAT2 protein (p113) are closely associated with the intracellular domain of the IFNAR1 chain of the Type 1-IFN receptor (Abramovich et al. EMBO 1994, 13, 5871-5877).

Expression of the IFNAR1 chain in procaryotic and eucaryotic cells has enabled the preparation of a series of recombinant soluble proteins corresponding to the extracellular domain of IFNAR1, either as isolated native sequences or as fused proteins with the γ and κ chains of human IgG1, which are capable of acting as soluble receptors for type 1-IFN (Benoit et al. J. Immunol. 1993, 150, 707-716; see also published International Application WO 92/18626). Using such a soluble Type 1-IFN receptor, a Type 1-IFN receptor neutralising monoclonal antibody has also been obtained (monoclonal antibody 64G12, the hybridoma for which has been deposited at the European Collection of Cell Structures, formally the ECACC, under accession no. 92022605 (deposit made on 26 Feb. 1992 in the name of Laboratoire Europeen De Biotechnologie S.A. having its registered office at 28, Boulevard Camélinat-92233 Gennevilliers, France); see Published International Application WO 93/20187).This antibody and functionally equivalent antibodies have been proposed for use in treatment of autoimmune diseases such as rheumatoid arthritis.

Treatment of cynomologous monkeys with monoclonal antibody (mab) 64G12 has been reported to result in marked increase in skin allograft survival (Benizri et al. IFN & Cytokine Res. 1998, 18, 273-284). Treatment of cynomologous monkeys with mab 64G12 together with a sub-effective dose of cyclosporin A was also found to result in marked inhibition of graft-versus-host disease in animals grafted with allogenic bone marrow from histocompatibility class I and class II antigen divergent animals.

Macaques infected with simian immunodeficiency virus (SIV) are considered a useful model for the study of host factors which play a role in the development of AIDS. In this animal model, IFN-a production during primary infection is insufficient to prevent the establishment of a chronic infection and the development of immunodeficiency. This is followed by a second phase of IFN-α production similar to that observed in AIDS patients. It has been found that there is a close relationship between the presence of this interferon and the loss of CD 4⁺ cells which accompanies the development of the clinical signs of disease in SIV infected macaques. When such SIV-infected macaques with high levels of circulating IFN-α were administered mab 64G12, this resulted in a pronounced and prolonged increase in the level of circulating CD4⁺ cells (Khatissian et al. AIDS & Human Retroviruses 1996, 12, 1273-1278).

Earlier published International Applications in common ownership with the present application have disclosed that Type 1-IFN demonstrates anti-viral and anti-tumour activity when administered by the oromucosal route (WO 97/41883, WO 97/41884 and WO 97/41886).

### SUMMARY OF THE INVENTION

It has now additionally been found that anti-viral activity of IL-2 administered by the oromucosal route can be reduced by either intravenous or oromucosal administration of Type 1-IFN antibody. This is consistent with a previous finding that recombinant murine IL-2 can induce Type 1-IFN in vitro and opens the way for new therapeutic treatments for diseases associated with undesirable Th1 cell activity based on oromucosal administration of Th1 cytokine antagonists, for example administration of antibodies which bind to a Th1 cytokine such as Type 1-IFN or inhibit binding of such a cytokine at its receptor.

In one aspect, the present invention provides a method of inhibiting or treating a disease associated with action of a cytokine which stimulates or enhances a Th 1 cell response (a Th1 cytokine), said method comprising oromucosal administration of an antagonist of said cytokine. Such administration of a cytokine antagonist provides a new route for inhibiting or treating autoimmune diseases. In particular, for example an autoimmune disease associated with abnormal production or activity of Type 1-IFN may be treated by an oromucosal form of administration of a Type 1-IFN antagonist. Preferably, such an antagonist may be, for example, an antibody including an anti-Type 1-IFN receptor antibody or a non-cytokine receptor binding antibody which also does not bind a component of the signal transduction pathway of a cytokine receptor. More particularly, preferably a monoclonal anti-Type 1-IFN, anti-IFN-α or anti-Type 1-IFN receptor antibody may be employed.

In a further aspect, the present invention provides a pharmaceutical composition comprising a cytokine antagonist, e.g. an anti-Type1-IFN, anti-IFN-α or anti-Type 1-IFN receptor antibody, in a dosage form specifically adapted for oromucosal administration in accordance with a method of the invention.

### BRIEF DESCRIPTION OF THE FIGURES

FIG. 1 shows the effect of a polyclonal antibody against murine IFN-α/β administered by intravenous injection on the anti-viral effect of murine recombinant IL-2 given by oromucosal administration.

FIG. 2 shows the effect of polyclonal antibody against murine IFN-α/β administered by intravenous injection on the anti-viral activity of murine IFN-α/β or recombinant murine IL-2 given by oromucosal administration.

FIG. 3 shows the effect of a polyclonal antibody to murine IFN-α/β administered by an oromucosal form of administration on anti-viral activity of recombinant murine IL-2 given by oromucosal administration.

### DETAILED DESCRIPTION OF THE INVENTION

Definitions

The term “Th 1 cytokine” will be understood herein to refer to any cytokine associated with stimulation or enhancement of Th 1 cell responses. In particular, the term will be understood to include IL-2, IL-12, IL-15, IL-18, TNF-β, IFN-γ and Type 1-IFN or more specifically IFN-α.

A Th 1 cytokine antagonist refers to any agent capable of inhibiting the activity of a Th1 cytokine either by binding the cytokine or by interfering with its signalling pathway, e.g by binding to the cytokine receptor. The term encompasses proteinaceous antagonists including soluble receptors for Th1 cytokines corresponding to native cellular receptor sequences, or analogues thereof, or fusion proteins of such a native or analogue sequence, e.g. fused to the Fc fragment of human IgG1 which retain binding specificity for the cytokine. It will be understood to additionally include antibodies capable of binding a Th1 cytokine or inhibiting binding of such a cytokine at its native receptor. As indicated above, soluble receptors for Type 1-IFN derived from the IFNAR1 chain have previously, for example, been described as well as antibodies capable of inhibiting binding of Type 1-IFN to its native receptor. Other Th1 cell cytokine antagonists may, however, be employed.

“Type 1-interferon” is used herein in conventional manner to refer generically to interferons which bind to the IFN-α/β receptor as, for example, presented at the surface of Daudi cells. Type 1 interferon antibody as used herein thus refers to an antibody against both α and β IFNs, IFN ω and IFN τ e.g. an antibody capable of binding both murine α and β IFNs or both human α and β IFNs. Such an antibody may be an antibody to, for example, a human leucocyte interferon preparation (Mogensen et al.Acta Pathol. Microbiol. Scand. Sect. B Microbiol. Immunol. 1975, 83, 443-452)

The term “antibody” as used herein will be understood to encompass monoclonal antibodies including humanised antibodies, e.g. CDR-grafted antibodies, and human antibodies, e.g produced by phage-display technology. It will also be understood to encompass fragments of antibodies which retain the same binding specificity, e.g F(ab′)₂, and single chain antibodies (ScFvs).

The term “oromucosa” refers to the mucosa lining the oral and/or nasopharyngeal cavities. “Oromucosal administration” as used herein will thus be understood to mean administration of a particular Th 1 cell cytokine antagonist into the nasal or oral cavity so that the antagonist makes contact with the oromucosa.

In one aspect, the present invention provides use of an antagonist of a cytokine which stimulates or enhances a Th1 cell response for the manufacture of a composition for oromucosal administration of said antagonist to inhibit or treat a disease associated with action of said cytokine. More particularly, the invention provides such use of a Th1 cytokine antagonist for inhibiting or treating an autoimmune disease. The autoimmune disease may be for example any autoimmune disease in which abnormal production or activity of IFN-α has been implicated as involved in the disease process, e.g. systemic lupus erythematosus, rheumatoid arthritis, type I diabetes, multiple sclerosis and psoriasis.

The Th1 cytokine antagonist may preferably be an antibody, especially for example an antibody which is an antagonist of Type 1-IFN or more specifically IFN-α. Such an antagonist may be preferably an antibody to Type 1-IFN or more specifically IFN-α.

Thus in a preferred embodiment,. the present invention provides use of an anti-Type 1-IFN, anti-IFN-α or anti-Type 1 IFN receptor antibody in the preparation of a composition for oromucosal administration for inhibiting or treating a disease associated with abnormal production or activity of Type 1 interferon or IFN-α, more particularly, for example an autoimmune disease such as an autoimmune disease as listed above.

Compositions of an Th1 cytokine antagonist for use in accordance with the invention by oromucosal administration, e.g compositions of an anti-Type 1-IFN or anti-IFN-α antibody, may be provided in any form suitable for intake via the nasal or oral route. Such a composition may be in the form of a liquid suitable for ingestion or inhalation, e.g a liquid suitable for administration by nasal spray or as nasal drops or an ingestible syrup. The composition may be in the form of a gel or paste. It may be in a solid dosage form such as a tablet or lozenge or capsule. It may be formulated for controlled-release of the antagonist in the oral or nasal cavity.

As hereinbefore indicated, in a further aspect the present invention thus additionally provides a pharmaceutical composition comprising a Th1 cytokine antagonist in a dosage form specifically adapted for oromucosal administration, including conveniently solid dosage forms for oral ingestion such as a tablet or as a liquid formulation packaged for nasal administration e.g. as a spray or drops.

The dosage will depend on the particular type of antagonist and the disease to be treated. Repeated administration of the antagonist over a period of time may carried out.

The following examples illustrate the invention.

### EXAMPLES

(i) Compositions

Natural Murine IFN-α/β

Murine IFN-α/β (murine Type 1-IFN) was prepared from cultures of C243-3 cells induced with Newcastle Disease Virus (NDV) and purified as described by Tovey et al., Proc. Soc. Exp. Biol and Med., 1974, 146, 809-815. The preparation had a titre of 1×10⁸ IU/ml and a specific activity of 5×10⁷ IU/mg protein as assayed on mouse L929 cells challenged with Vesicular Stomatitis virus (VSV) and standardised against the international reference preparation of murine IFN-α/β (G-002-9DD4-5411) of the U.S. National Institutes of Health.

Recombinant Murine IL-2

Recombinant murine IL-2 was purchased from R & D Systems Inc. The preparation was greater than 97% pure as determined by SDS-PAGE and had an ED₅₀ of 0.1 to 0.4 ng/ml measured in a cell proliferation assay using an IL-2 dependent murine cytotoxic T-cell line, CTLL-2 (Gearing and Bird, Lymphokines and Interferons, A Practical Approach, Clements et al. ed., IRL Press 1987, p. 296).

The recombinant murine IL-2 was combined with bovine serum albumin/phosphate buffered saline (BSA/PBS) excipient prior to administration.

BSA/PBS Excipient

Bovine serum albumin fraction V RIA grade, immunoglobulin free (catalogue no. A7888, Sigma, Mo., US) was dissolved at a final concentration of 100 μg/ml in phosphate buffered saline (pH 7.4) and sterile filtered (0.2μ).

Polyclonal Anti-Murine Type 1-IFN Antibodies

A sheep was immunised by subcutaneous injection with murine IFN-α/β at a titre of 1.6 to 8×10⁵ IU/ml in alum adjuvant. The sheep was then injected twice, with a ten day interval between the first and second injections, with 8×10⁶ IU of murine IFNα/β admixed with Freund's complete adjuvant as described in Gresser et al, J. Explt. Medicine, 1976, 144, 1305-1315. The neutralising titre of the anti-interferon anti-serum so obtained was 2.4×10⁶ against 10 IU of murine IFN-α/β. The crude antiserum was then adsorbed repeatedly on packed mouse cells, ultracentrifuged and precipitated with ammonium sulphate (Gresser et al.,ibid) to give a purified immunoglobulin fraction containing 44 mg/ml of IgG.

In the studies described below, polyclonal anti-murine Type 1-IFN antibody was employed at a neutralising titre of 4×10⁵ against 10 IU of murine Type 1-IFN or 3.2×10⁵ against 8 IU of murine Type 1-IFN.

(ii) Oromucosal Administration

Preliminary experiments showed that the application of 5 μl of crystal violet to each nostril of a normal adult mouse using a P20 Eppendorf micropipette resulted in an almost immediate distribution of the dye over the whole surface of the oropharyngeal cavity. Staining of the oropharyngeal cavity was still apparent some 30 minutes after application of the dye. Essentially similar results were obtained using ¹²⁵I-labelled recombinant human-α 1-8 applied in the same manner. The term oromucosal route as used below refers to administration of cytokine or antibody in the same manner.

(iii) Animals and Virus Infection

Groups of 10 6 week-old male Swiss mice from a pathogen-free breeding colony were infected with 100 LD50 of Encephalomyocarditis virus (EMCV; strain JH propagated on mouse L929 cells as described in Gresser et al., Proc. Soc. Exp. Biol. Med., 1968, 127, 491-496.) Stock EMCV was stored at −70° C. Diluted EMCV was prepared immediately before use and was kept on ice or in a refrigerator prior to administration.

### Comparative Example 1

The effect of intravenous injection of a polyclonal antibody to murine Type 1-IFN on the anti-viral activity of recombinant murine IL-2 administered via the oromucosal route to mice infected with a lethal virus dose.

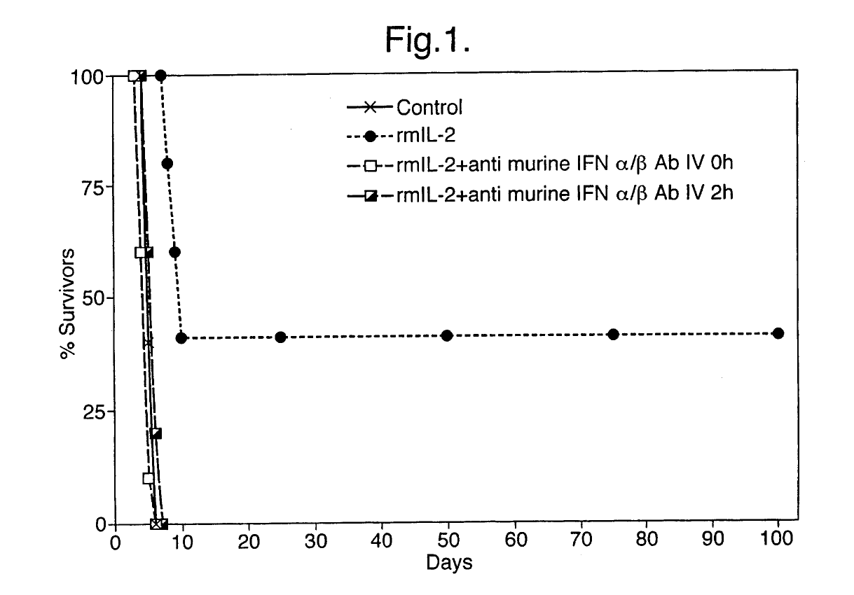

One hour after virus infection, mice were either left untreated or treated once a day for 4 days by the oromucosal route with 1.0 μg of recombinant murine IL-2 in 10 μl of BSA/PBS excipient, or with an equal volume of excipient alone. Alternatively, mice were treated by the oromucosal route with 1.0 μg of recombinant murine IL-2 in 10 μl of BSA/PBS excipient or with 10 μl of BSA/PBS excipient once a day for 4 days together with 200 μl of polyclonal murine Type 1-IFN antibody (neutralising titre 4×10⁵ against 10 IU of IFN-α/β) administered as a single intravenous injection at the time of virus infection. The results are presented in FIG. 1.

Results

Treatment of adult mice with recombinant murine IL-2 by the oromucosal route resulted in a marked increase in the percentage of animals surviving infection with a lethal dose of ECMV (survival of 30% of animals; IL-2 treated animals were alive and well 100 days after infection under conditions where all the untreated or excipient only treated animals were dead at 6 days). Intravenous injection of polyclonal anti-murine Type 1-IFN antibody at the time of virus infection resulted in enhanced virus replication and accelerated death in animals given excipient without IL-2 (5 days compared to 6 days). Treatment of animals with 1 μg of recombinant murine IL-2 together with a single intravenous injection of polygonal anti-murine Type 1-IFN antibody at the time of virus infection markedly inhibited the anti-viral activity of recombinant IL-2. Thus all the animals so treated were dead at 8 days after virus infection.

### Comparative Example 2

The effect of intravenous injection of polyclonal anti-murine Type 1-IFN antibody on the anti-viral activity of murine IL-2 or murine Type 1-IFN administered by the oromucosal route

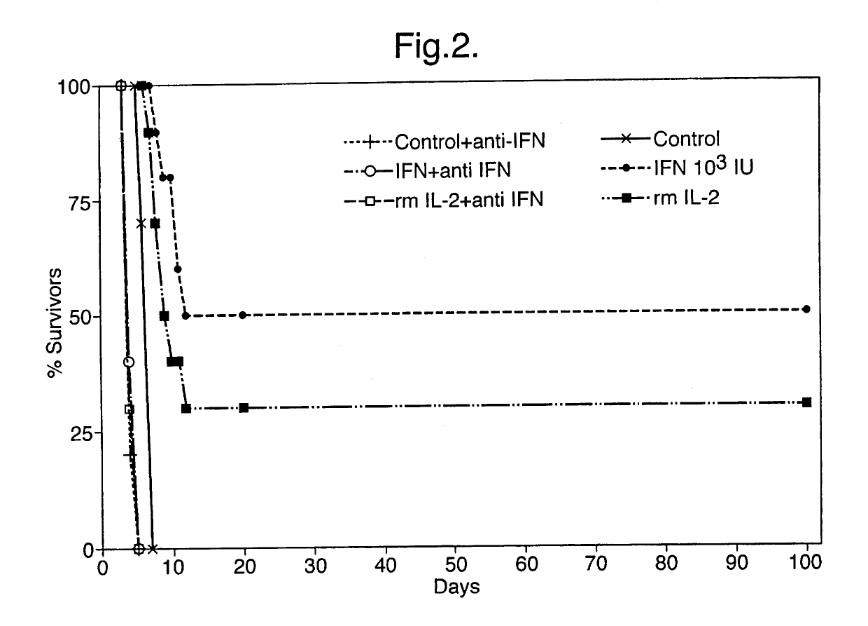

One hour after virus infection, mice were either left untreated or treated once a day for 4 days by the oromucosal route with 1.0 μg of recombinant murine IL-2 or 1000 IU of murine Type 1-IFN in BSA/PBS excipient or with an equal volume (10 μl) of excipient alone. Alternatively, mice were treated by the oromucosal route with 1.0 μg of recombinant murine IL-2 in 10 μl of excipient or with 1000 IU of murine Type 1-IFN in the BSA/PBS excipient or with 10 μl of excipient alone once a day for 4 days together with 200 μl of polyclonal anti-murine Type 1-IFN antibody (neutralising titre 4×10⁵ against 10 IU of murine Type 1-IFN) administered in a single intravenous injection at the time of virus infection. The results are presented in FIG. 2.

Results

As in comparative example 1, treatment of adult mice with recombinant murine IL-2 by the oromucosal route resulted in a marked increase in the percentage of animals surviving (30% survival 100 days after virus infection). Adult mice treated with murine Type 1-IFN also showed a marked increase in survival after the same period (50% survival ). Both the IL-2 and IFN-treated animals were alive and well after 100 days under conditions where all the untreated or excipient only treated animals were dead at 6 days. Intravenous injection of polyclonal anti-murine Type 1-IFN antibody at the time of virus infection resulted in enhanced virus replication and accelerated death of animals treated additionally only with excipient (all dead at 5 days compared with 7 days for animals treated with excipient alone). In animals given both recombinant murine IL-2 and anti-Type 1-IFN antibody, the anti-viral effect of the IL-2 was abrogated (all animals dead at 5 days after virus infection). Intravenous injection of anti-murine Type 1-IFN antibody similarly abrogated the anti-viral effect of murine Type 1-IFN administered by the oromucosal route.

### Example 3

Effect of polyclonal anti-murine Type 1-IFN antibody given by the oromucosal route on the anti-viral effect of recombinant murine IL-2 also given by the oromucosal route

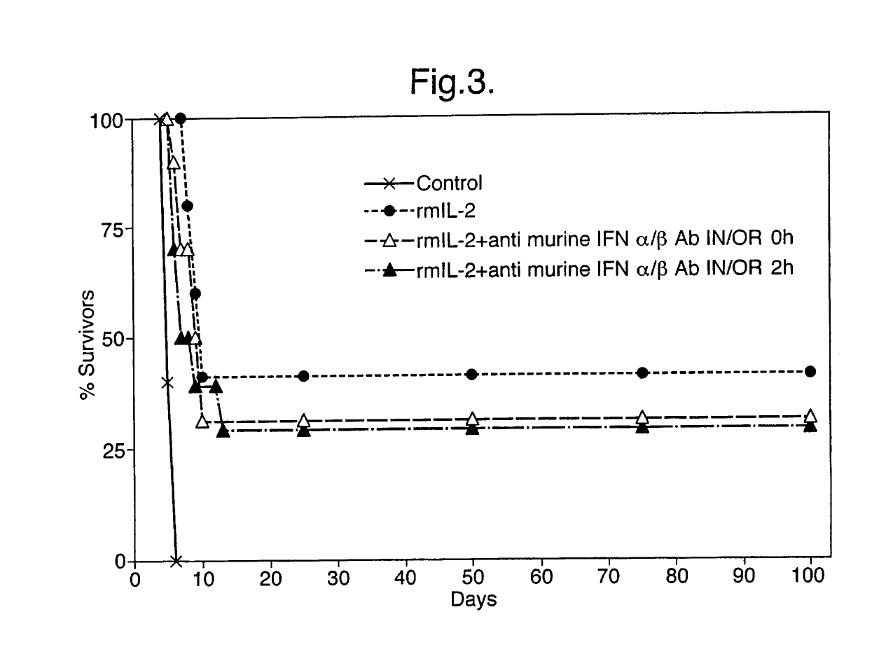

Immediately following virus infection, mice were treated once a day for 4 days by the oromucosal route with 1.0 μg of recombinant murine IL-2 in 10 μl of BSA/PBS excipient or with 10 μl of excipient alone. Alternatively, mice were treated by the oromucosal route with 1.0 μg of recombinant murine IL-2 in 10 μl of BSA/PBS excipient once a day for 4 days together with 20 μl of polyclonal anti-murine Type 1-IFN antibody (neutralising titre 3.2×10⁵ against 8 IU of murine Type 1-IFN) administered by the oromucosal route either immediately prior to or 2 hours after the administration of murine IL-2. The results are presented in FIG. 3.

Results

Treatment of adult mice with recombinant murine IL-2 by the oromucosal route resulted in a marked increase in the percentage of animals surviving infection with a lethal dose of EMCV (40% alive 100 days after virus infection under conditions where all the animals given excipient only were dead at 6 days). Treatment of animals with anti-murine Type 1-IFN antibody by the oromucosal route immediately prior to administration of recombinant murine IL-2 also by the oromucosal route markedly reduced the anti-viral activity of the IL-2 (30% of animals alive after 39 days). Treatment of animals with anti-murine Type 1-IFN antibody by the oromucosal route 2 hours after administration of recombinant murine IL-2 by the same route resulted in a similar degree of inhibition of anti-viral activity exhibited by the IL-2.

These results are consistent with IL-2 administered by the oromucosal route inducing Type 1-IFN and show that a Type 1-IFN antibody is a candidate for reducing Th1 cell over-reactivity in vivo also via oromucosal administration.

1. Use of an antagonist of a cytokine which cytokine stimulates or enhances a Th 1 cell response for the manufacture of a composition for oromucosal administration of said antagonist to inhibit or treat a disease associated with action of said cytokine.

2. The use of an antagonist as claimed in claim 1 wherein said disease is an autoimmune disease.

3. The use of an antagonist as claimed in claim 1 or claim 2 wherein said antagonist is an antibody.

4. The use of an antibody as claimed in claim 3 wherein said antibody is a Type 1-interferon (IFN) antagonist which does not bind to a cytokine receptor or a component of the signal transduction pathway of a cytokine receptor.

5. The use of an antibody as claimed in claim 5 wherein said antibody is a monoclonal anti-Type 1-IFN or anti-IFN-α antibody.

6. The use of an antibody as claimed in claim 3 wherein said antibody is an anti-Type 1-IFN receptor antibody.

7. The use of an antibody as claimed in any one of claims 4 to 6 wherein said autoimmune disease is an autoimmune disease associated with abnormal production or activity of IFN-α selected from systemic lupus erythematosus, rheumatoid arthritis, type 1 diabetes, multiple sclerosis and psoriasis.

8. The use of an antagonist as claimed in claim 1 or claim 2 wherein said antagonist is a soluble cytokine receptor corresponding to a native cellular receptor sequence, an analogue thereof or a fusion protein of such a native or analogue sequence which retains binding specificity for said cytokine.

9. A pharmaceutical composition comprising a cytokine antagonist as defined in claim 1 in combination with a pharmaceutically acceptable carrier or excipient, said composition being in a dosage form specifically adapted for oromucosal administration in accordance with any one of claims 1 to 8.

10. A pharmaceutical composition as claimed in claim 9 in a solid dosage form for oral ingestion.

11. A pharmaceutical composition as claimed in claim 9 which is a liquid formulation packaged for nasal administration.

12. A method of inhibiting or treating a disease associated with action of a cytokine which stimulates or enhances a Th 1 cell response, said method comprising oromucosal administration of an antagonist as defined in any one of claims 1 to 8.
